(19) United States
(12) Patent Application Publication
(10) Pub. No.: US 2016/0184029 A1
(43) Pub. Date: Jun. 30, 2016

(54) METHODS AND SYSTEMS FOR ADAPTIVE 3D IMAGING-GUIDED SINGLE-CELL MEASUREMENT

(71) Applicant: Allen Institute, Seattle, WA (US)

(72) Inventors: Hanchuan Peng, Bellevue, WA (US); Brian Russell Long, Seattle, WA (US); Lu Li, Bellevue, WA (US)

(21) Appl. No.: 14/984,236

(22) Filed: Dec. 30, 2015

Related U.S. Application Data
(60) Provisional application No. 62/098,443, filed on Dec. 31, 2014.
(60) Provisional application No. 62/164,116, filed on May 20, 2015.

(51) Int. Cl. A61B 34/20; A61B 5/04; A61B 5/00; A61B 34/10

(52) U.S. Cl. CPC A61B 34/20; A61B 34/10; A61B 5/04; A61B 5/6847; A61B 2034/107; A61B 2034/2055

(57) ABSTRACT

Systems and methods to facilitate automated image-guided experiments including in vivo electrophysiology and electroporation are disclosed. Exemplary systems and methods utilize three-dimensional image data to estimate coordinates of target cells and calculate a path for a probe to carry out an initial approaching toward a target location. Visualization and real-time image analysis is then employed to modify the path based on updated three-dimensional data of the probe location and target location, compensating displacement of target location due to the insertion of the probe into the tissue. Precise control of the probe then performs final approaching of the probe to the target location. This adaptive pipette positioning technique provides a platform for future advances in automated in vivo experiments.

### CROSS-REFERENCE TO RELATED APPLICATIONS

This applications claims the priority benefit, under 35 U.S.C. §119(e), of U.S. Application No. 62/098,443, filed Dec. 31, 2014, and of U.S. Application No. 62/164,116, filed on May 20, 2015, each of which is incorporated herein by reference in its entirety.

### BACKGROUND

Two-photon (2P) and related laser scanning microscopy methods have become powerful tools for deep-tissue imaging, particularly for in vivo studies of the nervous system. Since neuronal cells exhibit broad diversity, fluorescent labeling schemes can be used to target specific, genetically-defined neuronal subtypes. In addition to monitoring cell morphology and development, laser scanning imaging can also be used to target specific cells for monitoring of electrical activity, single-cell electroporation, or opto-genetics. To utilize two-photon or any similar laser scanning imaging technologies to probe individual cells, pipettes or other types of probes are typically inserted into the tissue and guided to the vicinity or in direct contact with the target cell. In current technologies, guiding of the pipettes is usually carried out manually, i.e., an operator manipulates the pipette to reach the target location.

### SUMMARY

The inventors have recognized that there is a need in the art for an automated, image-guided tool for single-cell measurements and manipulation. Accordingly, embodiments of the present invention include methods and systems for adaptive three-dimensional image-guided single cell measurement. In one exemplary embodiment, a method of positioning a distal end of a probe with respect to a target location in a tissue starts from estimating three-dimensional coordinates of the target location and the distal end of the probe in the tissue from a first image of the tissue. Then a processor estimates a path for the distal end of the probe to a desired location in the tissue based on the three-dimensional coordinates of the target location. An actuator moves the distal end of the probe to within about 25 μm of the three-dimensional coordinates of the target location along the estimated path, after which an imager acquires a second image of the target location and the distal end of the probe. (In practice, the 25 μm distance to the target location may correspond to a distance of about 60 μm from the center of a cell.) The processor uses the second image to estimate at least one change in the three-dimensional coordinates of the target location due to insertion and/or movement of the distal end of the probe into the tissue. With the change in the three-dimensional coordinates of the target location, the processor determines at least one change in the path from the distal end of the probe to the desired location in the tissue so as to allow the probe to approach the target location.

In another exemplary embodiment, a system includes a probe, an actuator, an imager, and a processor. The probe has a distal end to be inserted into a tissue. The actuator is mechanically coupled to the probe to move the distal end of the probe along a predetermined path to a desired location in the tissue. The desired location is within about 25 μm of a target location within the tissue. The imager is configured to acquire an image of the target location. The processor is operably coupled to the actuator and to the imager to estimate a change in position of the target location caused by insertion and/or movement of the distal end of the probe into the tissue and to determine at least one change in the predetermined path from the distal end of the probe to the desired location in the tissue based at least in part on the change in position of the target location.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 shows a schematic view of a probe positioning system including a processor to estimate location change of target cells in a tissue due to insertion of the probe into the tissue.

FIGS. 2A-2C illustrate a method of probe positioning in a tissue including steps of probe path modification during the approaching the probe toward desired locations.

FIG. 3 is a flowchart of a probe positioning method that includes probe path modification as the probe approaches a desired location in the tissue.

FIGS. 4A-4D are images of pipette tip and target cells acquired during probe positioning using two-photon laser scanning microscopy.

FIGS. 5A-5B are plots that illustrate the precision of virtual-finger single-click localization of pipette tip and cell positions.

FIGS. 6A-6C show final distance variations in automatic probe positioning approaches without image-guided adaptive pipette movement.

FIG. 7 shows final pipette positions relative to target locations in vivo and in vitro with image-guided adaptive pipette movement.

FIGS. 8A-8D show variability in pipette location in Cartesian coordinates and pipette-resolved components.

FIG. 9 shows total time for adaptive pipette approaches to complete the positioning.

FIGS. 10A and 10B illustrate images and EEG measurements, respectively, made using the system of FIG. 1.

### DETAILED DESCRIPTION

One goal in neuroscience is to understand how the activities and connections of individual neurons give rise to animal behavior. A feasible approach of achieving this goal is to measure the activity of individual neurons in intact, functioning cortical circuits. In practice, optical physiology measurement may be employed to monitor cell activities. Alternatively, electrophysiology measurements, such as whole-cell or cell-attached recordings, may also be used due to their high temporal resolution and/or sensitivity. Typically, exploring neuronal function in intact circuits can benefit from experimental modalities that provide both visualization and physical access to targeted cells.

Scanning two-photon (2P) microscopy and similar laser scanning microscopy techniques allow imaging of fluorescently-labeled biological structures, which can be hundreds of microns deep in scattering tissue. When combined with mouse lines expressing fluorescent proteins in genetically-defined cell types, 2P microscopy can visualize neuronal (sub)types and layer-specific populations deep in the neocortex of a living mouse. These labeled cells can then become a target for a wide range of measurements and manipulation, including optical and electrical recordings of cellular activity, driving expression of exogenous proteins using single-cell electroporation and targeted activation or inhibition with optogenetics. Single-cell electrophysiology measurements during sensory processing and behavioral tasks can be combined with gene expression for post-hoc labeling of the target cell or synaptically-connected partners.

Several practical challenges are associated with approaching a targeted cell with a physical probe and can make these experiments difficult for most researchers. Firstly, the manual process of bringing a probe in close proximity to a target single cell normally must be performed by an operator with extensive expertise to carefully control micromanipulators or similar apparatus.

Secondly, it can be challenging to achieve precision and stability in the guiding or positioning process so as to reduce or even eliminate lateral (off-axial) movements of the probe in brain tissue. Lateral movements of the probe may induce mechanical deformation of brain tissue and disruption of the neurites of target and neighboring cells.

Thirdly, it is generally desirable to have efficient and reproducible guiding of probes to the target cell(s), at least because multiple insertions into the brain may lead to brain inflammation and edema, conditions under which reliable measurements can be difficult. Manual guiding, however, normally does not possess the desired level of efficiency and reproducibility.

Lastly, cell-attached and whole-cell recordings, which can offer physical access to the cell, can be sensitive to the condition of the glass microelectrode (pipette) tip at the membrane of the target cell. During the guiding process, also referred to as positioning process or targeting process, increasing the number of penetrations and the amount of movements can also increase the likelihood of tip condition degradation, thereby potentially inducing the disruption of the tissue. Serious tissue disruptions, such as bleeding and brain swelling, are typically undesirable.

The conventional process of approaching a targeted cell typically can involve careful manual control of both the pipette micromanipulator and the microscope objective, as well as visual monitoring of pipet resistance and adjustment of the fluid flow from the pipet by manual pressure control. This can be a specialized process, in which months to years of training and practice may be performed to achieve proficiency. Moreover, targeted in vivo electrophysiology experiments can have low yield, even with a high level of expertise, especially for whole cell recordings.

Systems for Positioning Probes for Single-Cell Measurements

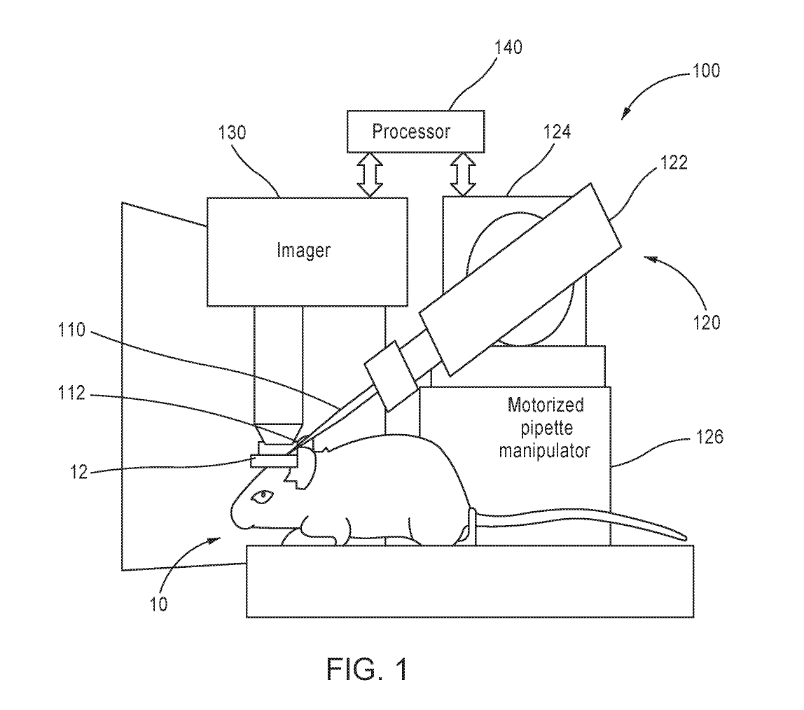

The above mentioned challenges in single-cell probing may be addressed, at least partially, by 3D image analysis and computerized probe control, e.g., by integrating volumetric image information and pipette control into a suite of 2P targeted experiments, as shown in FIG. 1. To be more specific, FIG. 1 shows a system 100 that addresses at least two phases in typical probe-based, 2P-targeted experimental techniques. The first phase is selecting a target cell and identifying the 3-dimensional (3D) location (3D coordinates). The second phase is bringing the pipette in close proximity to the target cell. By adaptively planning and executing the pipette trajectory based on 3D image data, the system 100 in FIG. 1 can robustly approach targeted cells while reducing lateral movement in the brain comparing to conventional manual techniques.

The system 100 in FIG. 1 includes a probe 110, an actuator 120, an imager 130, and a processor 140. The probe 110 has a distal end 112 that can be inserted into a tissue 12 of, for example, a mouse 10 or other living organism. The actuator 120 is mechanically coupled to the probe 110 to move the distal end 112 of the probe 110 along a predetermined path to a desired location in the tissue 12. The desired location is typically within about 25 μm of a target location within the tissue 12. The imager 130 is employed to acquire an image of the target location. The processor 140, which is operably coupled to the actuator 120 and to the imager 130, is configured to estimate a change in position of the target location caused by, for example, insertion and/or movement of the distal end 112 of the probe 110 into the tissue 12 and to determine at least one change in the predetermined path from the distal end 112 of the probe 110 to the desired location in the tissue 12 based at least in part on the change in position of the target location.

The probe 110 can be a pipette, also referred to as a pipet, pipettor, or chemical dropper. The probe 110 can have differing levels of accuracy and precision to accommodate different applications. For example, the probe 110 can be single piece glass pipettes to more complex adjustable or electronic pipettes. Pipettes for scientific use are typically made of glass, e.g., borosilicate. Here, a pipette can be fabricated by pulling a capillary while heating until it breaks to form a tapered, fine point (with a tip inner diameter of approximately 1 micron). Other possible probes include but are not limited to fine metal wires, silicon with metal electrodes microfabricated onto the surface, and fiber optic devices. These probes could be labeled in some way to be visible in whatever imaging modality is used.

In one example, the probe 110 can be configured for passive measurement (i.e., without actively disrupting the tissue cells), such as sensing an electrical signal at the distal end 112 of the probe 110. In another example, the probe 110 can be configured to convey matter into the tissue 12 via the distal end 112 of the probe 110, such as medicine, labeling chemicals, etc. In yet another example, the probe 110 can be configured to withdraw matter from the tissue 12 via the distal end 112 of the probe 110 so as to, for example, study the composition or behavior of the cells in the tissue 12. In yet another example, the probe 110 can be configured to physically disrupt the tissue 12 via either the distal end 112 or other portion of the probe 110. In yet another example, the probe 110 can be configured to emit light towards the target (optrodes) for optogenetic stimulation and fluorescence measurement. In yet another example, the probe 110 can be configured as an electrode for extracellular recordings, in which the probe 110 can have multiple recording sites along the probe. In other words, the distal end 112, as well as other portions of the probe 110, can both be employed as recording sites. In yet another example, the probe 110 can be configured to perform one or more of the above mentioned tasks.

In one example, the actuator 120, as shown in FIG. 1, includes an axial part 122, a lateral part 124, and a support structure 126. In operation, the axial part 122 can be configured to move the probe 110 along the axial direction of the probe 110, and the lateral part 124 can be configured to move the probe 110 along a lateral direction, which can be perpendicular to the axial direction. If desired, the lateral and axial movements can also be driven by the same actuator (e.g., a Sutter MP-286 micromanipulator). Suitable actuators includes, but are not limited to piezos, galvos, stepper motors, and voice coils that can be controlled by a computer or other processor-based instrument with sufficient precision and accuracy to operate across hundreds of microns with 0.1-micron precision or better. The support structure 126 can be, for example, a pedestal to position the axial part 122 and lateral part 124 at an appropriate height. In one example, the pedestal 126 can have a fixed height. In another example, the pedestal 126 can have the option to move vertically so as to add another degree of freedom into the probe movement.

In another example, the actuator 120 can be configured to perform three-dimensional positioning of the probe 110. Three-dimensional positioning can be achieved by, for example, including a second lateral movement unit into the pedestal 126. The second lateral movement can be perpendicular to both the axial direction and the lateral direction enabled by the lateral part 124. Alternatively or in addition, three-dimensional positioning can be achieved by integrating a second lateral movement unit into the axial part 122. For example, the second lateral movement part in the pedestal 126 can be configured for coarse movement, while the second lateral movement part in the axial part 122 can be configured for fine movement. The actuator may also enable rotational movement and alignment, e.g., for pitch, yaw, and roll.

The actuator 120 can be configured to have different movement precisions depending on, for example, the point of interest to be probed. For example, if the point of interest is a single cell, the actuator 120 can be configured to provide lateral alignment within about 3 microns (in vitro testing) or 5 microns (in vivo testing), and radial (axial) alignment within about 4 microns (in vitro testing) and 8 microns (in vivo testing). In theory, this technique can be applied to any cell that can be visualized by the imager. For instance, one could also target blood vessels in the brain for drug delivery or other purposes.

The imager 130 in the system 100 is configured to acquire an three-dimensional image or volumetric image of at least a portion of the tissue 12 so as to facilitate the movement of the probe 110 to the desired location. The desired location is typically within 25 microns of a target location within the tissue 12. For example, the target location can be a collection of locations of cells of interest (e.g., neuron cells), and the desired location can be a location in the proximity of any one of the cells of interest.

In one example, the imager 130 can utilize the two-photon laser scanning microscopy technique and sense fluorescence generated by stimulating at least one fluorophore in the tissue. Other suitable imaging modalities include infrared differential interference contrast (DIC) images, e.g., for applications in reduced tissue preparation, such as brain slices. Third-harmonic generation, coherent anti-stokes Raman (CARS), and stimulated Raman scattering are other microscopy methods that can image in 3D deep in tissue.

The imager 130 can be configured to image different portion of the tissue 12 or other components in the system 100. For example, the imager 130 can be configured to image the target location and the distal tip 112 of the probe 110 so as to allow the calculation of probe path toward the desired location. The imager 130 can also be configured to image the desired location closely (e.g., with a smaller field of view) so as to allow the accurate positioning of the distal end 112 of the probe 110. Moreover, the imager 130 can be configured to image the tissue 12 and the distal end 112 of the probe 110 separately. For example, the imager 130 can acquire one image for the target location and another image for the distal end 112 of the probe 110 with higher resolution than that in a single image.

The imager 130 can be configured to have different spatial resolutions depending, for example, on the dimensions of the cells to be probed. For example, larger cells may allow the use of lower spatial resolution as readily understood in the art. Moreover, the spatial resolution can also depend on the location of the distal end 112 of the probe 110 with respect to the desired location. For example, when the distal end 112 is far away from the desired location, the movement can have a large step size and the resolution of the image can be low. On the other hand, when the distal end 112 is in the proximity of the desired location, it may be beneficial to have a high resolution so as to accurately position the distal end 112 to the desired location. A practical range of spatial resolution of the imager 130 can be about 1 μm to about 5 μm, or 2 μm to 4 μm.

The processor 140 in the system 100 is operably coupled to the actuator 120 and the imager 130. The communication between the processor 140 and the imager 130, and the communication between the processor 140 and the actuator 120, can be bidirectional. The processor 140 can take information from the imager 130 so as to, for example, identify a desired location, calculate a path for the distal end 112 of the probe 110 to reach the desired location, or make adjustment of the calculated path based on an updated image from the imager 130. The processor 140 can then instruct the actuator 120 to move the distal end 112 of the probe 110 according to the calculated path. The processor 140 can also take information from the actuator 120, such as the speed of probe movement, the step size of the probe movement, or the angle of the probe with respect to the surface of the tissue 12, among others. This information can be fed back to the imager 130 to, for example, compare the projected location of the distal end 112 of the probe 110 and the actual location, allowing changes in the probe path in a real-time or near real-time manner.

The processor 140 can be configured to estimate the probe path based on a variety of parameters, including initial location, target location, scale factors of the images, step size of the intended probe movement, speed of the intended movement, and probe angle, among others. In operation, the target location may change due to the insertion of the probe 110, the movement of the mouse 10, or both. The processor 140 can be configured to estimate the initial and adjusted probe paths either taking into account the motion of the target location or without accounting for the motion of the target location. For example, when the distal end 112 of the probe 110 is still far away (e.g., >50 μm) from the desired location, the processor 140 can be configured to estimate the probe path without considering the possible motion of target location.

The system 100 shown in FIG. 1, in general, can be configured to move the distal end 112 of the probe 110 to the desired location in the tissue 12 within about 10 minutes of inserting the probe 110 into the tissue 12. This time scale can be comparable to manual operation of the probe positioning process. However, the system 100 can collect and store more image information, which can be used in subsequent analysis and path adjustment.

Moreover, compared to manual probe positioning, the speed of the probe movement may be adjusted more conveniently in the system 100 to complete the positioning of the probe 110 within different time constraints. In one example, the system 100 can be configured to move the probe at a constant speed (e.g., 3-4 μm per second) throughout the entire position process. In another example, the system 100 can be configured to move the probe at different speeds when the distal end 112 of the probe 120 is at different locations. For example, the speed can be larger when the distal end 112 is more than 50 μm away from the desired location, or more than 25 μm away from the desired location, or more than 15 μm away from the desired location. When the distal end 112 of the probe 110 is sufficiently close to the desired location (e.g., <50 μm, <25 μm, or <15 μm), the speed of probe movement can be reduced so as to achieve accurate positioning. For a given application, the probe speed/transit time may be comparable to that of human operator/experimenter. Speed deep within the brain may be about 0.1-5.0 μm/sec, but speeds above the brain or entering the brain could be as high as about 1.0 mm/s.

Methods of Probe Positioning for Single-Cell Measurements

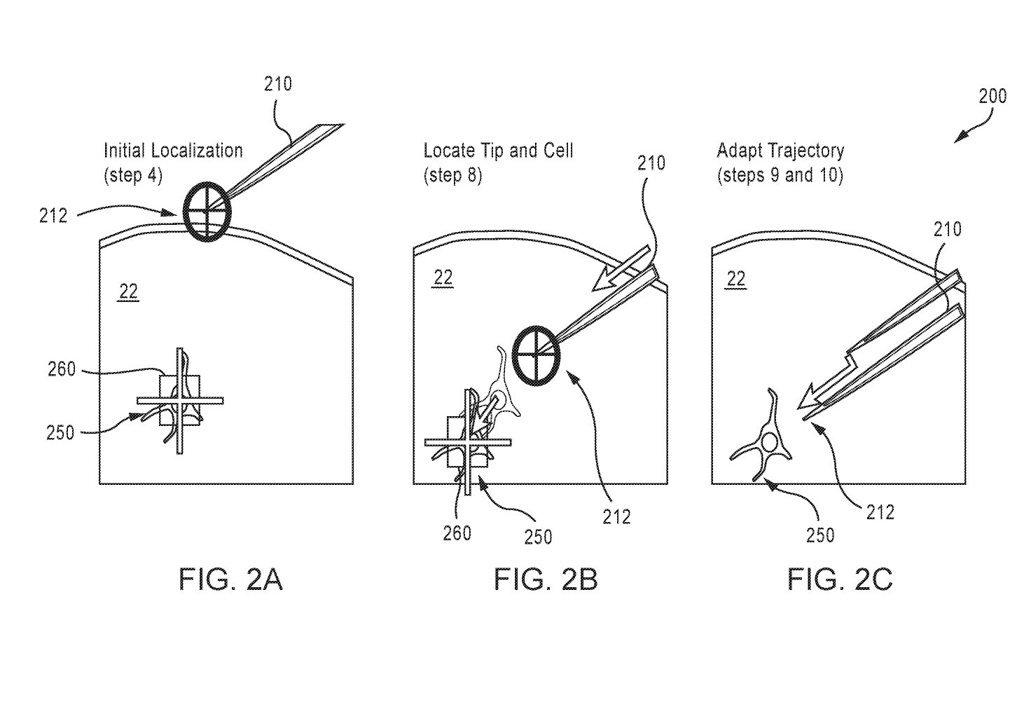

FIGS. 2A-2C illustrate a method 200 of guiding a probe to a desired location. This method 200 can be implemented by a system like the one shown in FIG. 1. In FIG. 2A, a distal end 212 of a probe 210 is placed at an initial location. The initial location can be, for example, above the surface of a tissue 22, or within the tissue 22 but far away from a target location 250. Only one target location is shown in FIGS. 2A-2C. However, in practice, the target location 250 can comprise a collection of center locations of cells. In this stage, three-dimensional coordinates of the target location 250 and the distal end 212 of the probe 210 are estimated based on a first three-dimensional image acquired by an imager (not shown). Based, at least in part, on the three-dimensional coordinates of the distal end 212 and target location 250, the processor calculates a path for the distal end 212 of the probe 210 to a desired location 260 in the tissue. The location of the distal end 212 is shown as a cross in a circle. The desired location 260 is shown as a cross in a rectangle, within which the distal end 212 of the probe 210 is to be positioned.

In FIG. 2B, the distal end 212 of the probe 210 is at a second location that is closer to the desired location 260 or the target location 250. The probe 210 is moved at least partially along the path estimated in FIG. 2A to arrive at this second location, which is within about 25 μm to 75 μm of the three-dimensional coordinates of the target location 250. The movement at this stage can be substantially axial, i.e., along the direction of the probe body, as shown by the arrow in FIG. 2B.

Upon arrival at the second location, a second three-dimensional image is acquired by the imager (not shown) to evaluate whether the target location 250 changes. In practice, the target location's position can be influenced by, for example, insertion of the probe 210, movement of the tissue 22, or other environmental factors. A new set of three-dimensional coordinates of the changed target location 250 can be estimated based on the second image, and at least one change in the three-dimensional coordinates of the target location 250 is identified. Using this change in three-dimensional coordinates of the target location 250, a new path for the distal end 212 of the probe 210 can be estimated, and at least one change in the path from the distal end 212 of the probe 210 to the desired location 260 in the tissue is identified.

With the modified probe path estimated in FIG. 2B, the distal end 212 of the probe 210 is then moved again toward the target location 250 until the distal end 212 is within the range of the desired location (not shown in FIG. 2C). FIG. 2C shows the location of the distal end 212 of the probe 210 upon the completion of the method 200. The modified probe path can include modification on both axial direction and lateral direction, as indicated by the arrow in FIG. 2C.

In the method 200, the precision of target locating can be determined by the resolution of the first image acquired by the imager, as well as the quality of tissue preparation (e.g., brain motion for in vivo prep). For example, the precision of the target location 250 can be about 2.3 μm in two-photon imaging using a Sutter Moveable Objective Microscope with a 40× objective (LUMPLFLN 40W) controlled by ScanImage 3.8, with an excitation wavelength of 920 nm. The maximum power of the excitation laser beam can be up to about 75 mW at the sample plane, with typical intensity for z stacks at 10-50% of that value. The recording headstage can be mounted to a Sutter MP-285 manipulator positioned so the pipette (probe) pointed anterior, oriented approximately 31 degrees down from horizontal. For widefield imaging, the surgical site can be illuminated with an endoscope and visualized with a color CCD camera.

Higher image resolution (e.g., <2 μm, <1.5 μm, or <1 μm) can also be used in estimating the three-dimensional coordinates. In general, higher resolution can lead to improved localization (pinpointing). For static images, the target location 250 can be precise in the sub-pixel resolution. However, for dynamic images (e.g., in vivo testing), the movement of the tissue 22 may render the location above a precision of 1 μm. Similar precision ranges can also apply to the location of the distal end 212 when the distal end 212 is inserted into the tissue 22.

The second location of the distal end 212 of the probe 210 as shown in FIG. 2B can be from about 25 μm to about 75 μm from the target location 250 without considering the motion of the target location 250. This distance range can be influenced by at least two factors. At the high end of the range, it is helpful for the distal end 212 of the probe 210 at the second location to be still within the field of view of the imager, such that the imager can still capture volumetric information of the distal end 212 and make correction to the probe path if needed. At the low end of the range, it can also be helpful for the distal end 212 of the probe 210 to stop moving before past the target location 250 estimated in FIG. 2A. Since no correction is normally before the distal end 212 of the probe 210 arrives at the second location, passing the initial target location 250 may lead to errors in probe positioning.

The speed of the probe movement in the method 200 can be dependent on specific applications. For example, in vivo applications normally operate with a low velocity so as to avoid and reduce tissue disruption. A practical range of movement speed can be 3-4 μm/s, although higher or lower speeds are technically feasible.

The first image and the second image, based on which three-dimensional coordinates are estimated, are both acquired by the imager. In practice, different imagers can be employed to obtain the first image and the second image. Or different resolutions can be preset when acquiring the first image and the second image. For example, the second image can have a higher resolution since the distal end 212 of the probe 210 is closer to the target location 250.

The modification of probe path in FIG. 2C can compensate for the motion of the target location 250 due to, for example, insertion of the probe 210 or movement of the tissue 22. In the latter case, when the tissue 22 moves when the distal end 212 of the probe 210 approaches the second location, at least two possible situations may occur. In the first situation, the target location of the target cell can move within the image and the probe can stay in the same place. In this case, the target location 250 can be modified or corrected without modifying the location of the distal end 212. In the second situation, when both the target cell and the probe 210 have moved, corresponding corrections of both the target location 250 and the location of the distal end 212 can be helpful. The modification of the target location 250 and/or the location of the distal end 212 of the probe 210 can be less than 20 μm, and are typically less than 10 μm.

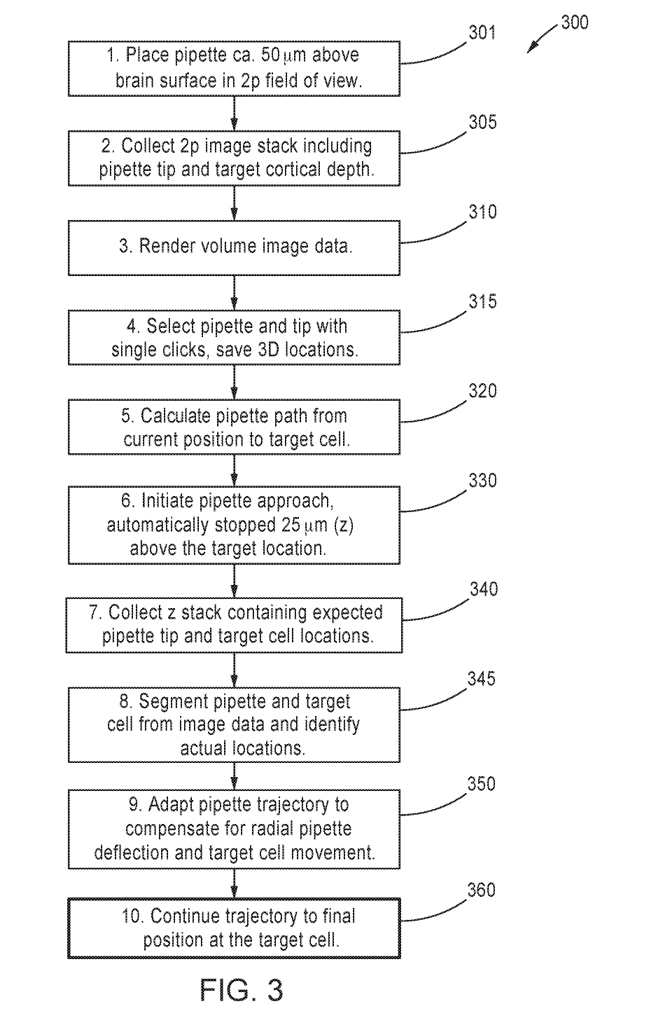

A more detailed method of positioning a probe (e.g., a pipette) for single-cell measurement is illustrated in FIG. 3 as a flowchart. In this process 300, a pipette is first placed about 50 μm above the tissue containing the target cells being investigated (step 301). The pipette tip and the target cells can be both within a field of view of an imager, such as a two-photon laser scanning microscope. In step 305, one or more two-photon image stacks can be collected. The image stacks include information at least about the pipette tip and the cortical depth of target cells. Based on the image stacks, volumetric data of the pipette tip and the target cells can be obtained in step 310. For example, the volumetric data can include three-dimensional coordinates of the locations of the target cells and the pipette tip. One target cell in the image can be selected and the location of the selected target cell can be set as the desired location, as in step 315. Using the pipette tip location as the initial location, and the desired location selected in step 315 as the final location, a pipette path can be calculated in step 320 in order for the pipette tip to approach the selected target cell.

The precision of target locating is largely determined by the resolution of the image and quality of tissue preparation (e.g. brain motion for in vivo prep). The target cell location precision is about 2.3 microns in some configuration, enabling the use of relatively low resolution scans to reduce acquisition times. Increased image resolution would improve localization, but target location is probably not reliable beyond about 1 micron in vivo because of brain movement. For static images, the target location is very precise in the sub-pixel resolution.

Following, at least partially, along the pipette path calculated in step 320, the pipette can move from its current location toward the desired location, until about 25 μm to about 75 μm away from the target location, as illustrated in step 330. The distance from the desired location may be limited by (1) still capturing the pipette tip in the scan volume at the high end and 2) at the low end, running into or past the target cell can pose issues as well. This initial approach automatically stops before reaching the target cell because motion of the brain tissue may occur at least due to the insertion of the pipette and/or the movement of the brain itself. Taking a pause at this stage allows the possibility to modify the calculated probe path and compensate for the movement of the brain tissue.

After the initial approach is completed, a second image containing expected pipette tip and target cells is collected in step 340. The second image allows the evaluation of whether any movement of target cell occurs. The second image also allows the comparison between the expected pipette tip location and the actual pipette tip location, thereby allowing corresponding correction. Step 345 segments the pipette and target cell from image data and identify their actual locations. Based on these actual locations, the pipette path or trajectory can be adapted, modified, or even recalculated, in step 350 so as to compensate for the movement of the target cells. The compensation can be on both radial and lateral directions. With the new pipette path, the pipette can be guided to the desired location (final position) in the proximity of the selected target cell.

In the method 300, the pipette can move at different speeds at different steps, For example, the pipette tip can move at 1300 μm/s before touching the brain surface. When the pipette tip is within the brain, the speed can be reduced to 6 μm/s during the first 40 μm into the brain. After 40 μm into the brain, the speed can be further reduced to, for example, 3-4 μm/s.

Exemplary Implementation of Probe Positioning for Single-Cell Measurement

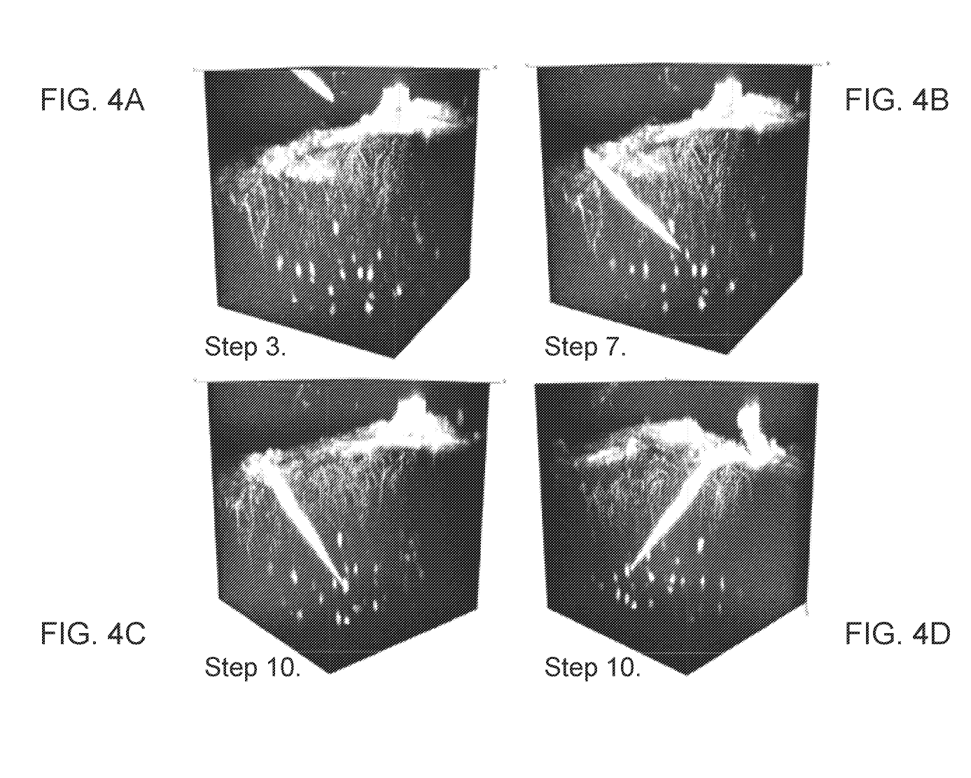

A software suite called smartACT (smart, Adaptive localization and Cell Targeting) is developed to implement the systems and methods described above. In this implementation, volumetric image information is employed for a pipette tip to adaptively approach a user-targeted cell in three-dimensional space. Analysis software includes Vaa3D (www.vaa3d.org), and custom analysis routines and user interfaces can be written in MATLAB, utilizing the microscope control and MP-285 driver available in ScanImage 3.8. Using a configuration for in vivo 2P imaging, 3D image data of fluorescently-labeled neurons in the mouse cortex can be collected, as shown in FIGS. 4A-4D.

The smartACT workflow can be described as follows:

1) Collect a scan containing the pipette tip and the neurons of interest. Then display 3D volumetric image data in Vaa3D, which allows instant easy 3D visualization of the surface of the brain and fluorescently labeled neurons in the neocortex, possibly limited by the depth of 2P scanning microscopy. A detailed description of Vaa3D can be found in the following papers, each of which is incorporated by reference herein in its entirety: Peng, H., et al., “V3D enables real-time 3D visualization and quantitative analysis of large-scale biological image data sets,” Nature Biotechnology, Vol. 28, No. 4, pp. 348-353, DOI: 10.1038/nbt.1612, (2010); Peng, H., et al., “Extensible visualization and analysis for multidimensional images using Vaa3D,” Nature Protocols, Vol. 9, No. 1, pp. 193-208, (2014); and Peng, H., et al., “Automatic reconstruction of 3D neuron structures using a graph-augmented deformable model,” Bioinformatics, Vol. 26, pp. i38-i46, (2010).

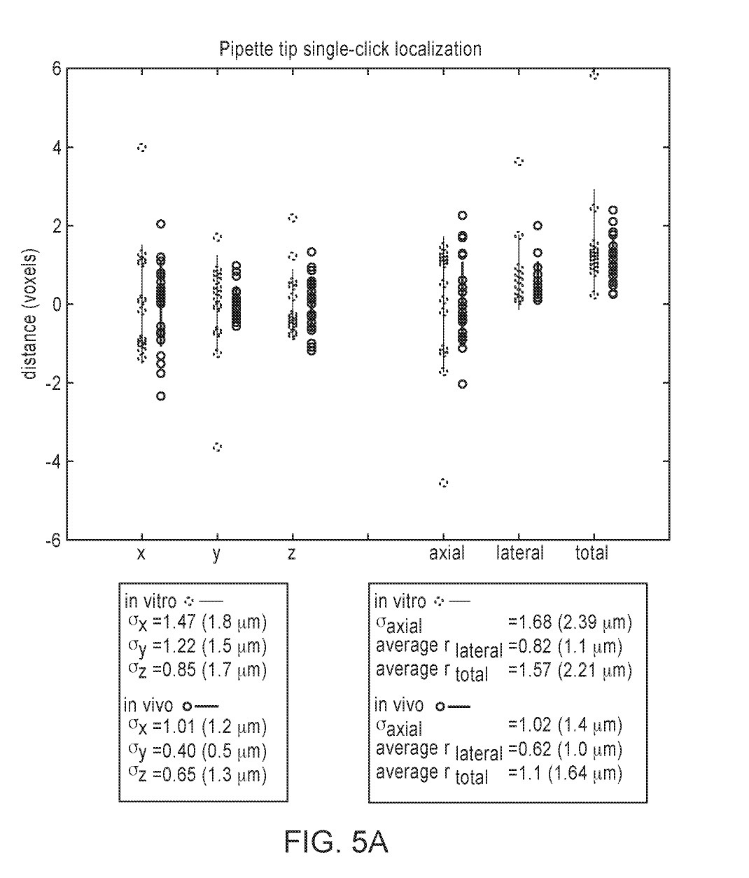

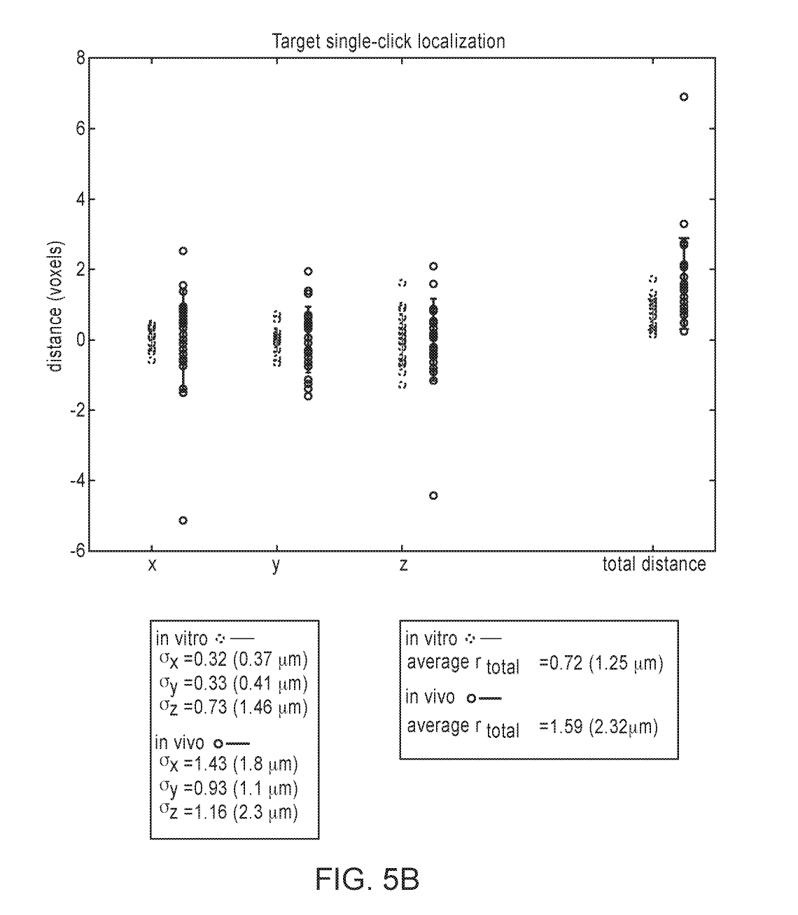

3D visualization and the ability to rotate the data in three dimensions and view the fluorescence signal from all angles allows rapid understanding of spatial relationships in the data useful for target selection. The implemented method of 3D target selection adapted from single computer-mouse operation (e.g., one mouse click) ‘virtual-finger’ technology can provide an intuitive interface for precisely locating the tip of the pipette and the center of the target cell. In the configuration with (1.23×1.23×2) μm voxel size, the target selection method can have a mean square deviation=2.32 μm from N=25 localizations, as shown in FIGS. 5A-5B, and yield accurate localization of the pipette tip and target cell within the image. FIGS. 5A-5B show N=13, 15 localizations of the labeled target cell in each of two experiments in vivo (green), N=11, 14 localizations of the pipette tip above the surface of the brain in the same image stacks (green), N=18 localizations of a 2 micron fluorescent bead in agarose (in vitro) (blue), N=14 localizations of the pipette tip in the same image stack (blue). All distances are from the mean position of the respective pipette or target localizations.

Additional description of ‘virtual finger’ technology can be found in Peng, H., et al., “Virtual finger boosts three-dimensional imaging and microsurgery as well as terabyte volume image visualization and analysis,” Nature Communication, DOI: 10.1038/ncomms5342 (2014), which is incorporated by reference herein.

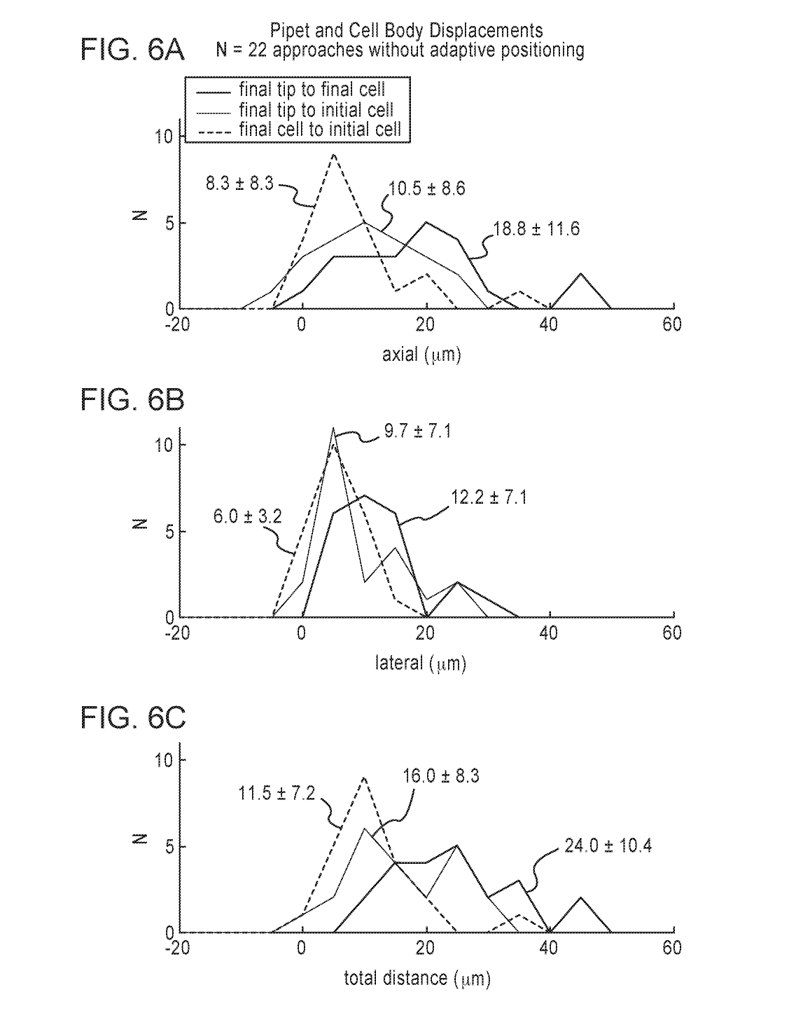

2) Calculate the path to the target cell. The path can approach along the pipette axis and terminate at a user-specified buffer radius from center of the target cell. The path can be subdivided into discrete steps, with the final approach through the cortex progressing in a sequence of axial movements. The size of these steps can be user-defined, with 2-4 μm steps used in the data presented in this specification. This method can position the pipette tip roughly in the vicinity of the target, as shown in FIGS. 6A-6C, with about 10 μm contributions of the error from both pipette deflection and target cell displacement during the approaching process. FIGS. 6A-6C show histograms of distance along the pipette axis (parallel), lateral to the pipette axis (perpendicular) and total distance for N=22 non-adaptive approaches in vivo. The distance between the final target position and the initial target position (red) quantifies cell body displacement during the approach. The distance between the final pipette tip position and the initial target position (green) quantifies pipette tip deflection during the approach. The distance between the final target position and the final pipette tip position (blue) quantifies the final separation at the terminus of the non-adaptive approach. The intended final position is displaced about 10-12 microns axially and 0 microns laterally from the center of the cell body.

This range of final positions may be traditionally accepted as sufficiently precise for 2P-targeted electrophysiology. However, it may not be sufficiently accurate for computerized control of the pipette positioning. More specifically, the lateral distance (closest distance from the target to the pipette axis) rₗₐₜₑᵣₐₗ is 12.2±7.1 μm, indicating that manual adjustment may be helpful to reach the target location on the surface of the cell, while the pipette is deep in the cortex.

3) In response to this variability in final pipette positions relative to the target cell, an adaptive method is developed. The adaptive method takes advantage of volumetric image data collected at an intermediate point along the route to the target. The smartACT method can reduce the axial (off-axis) displacement and refine the position of the pipette relative to the target cell.

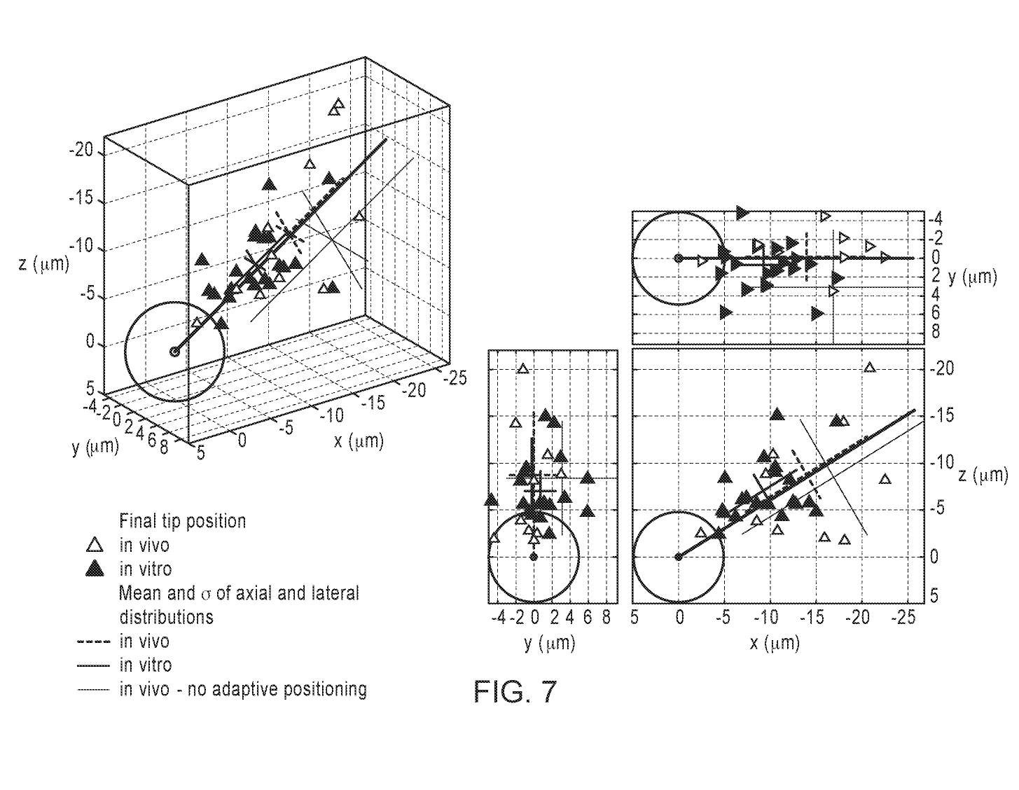

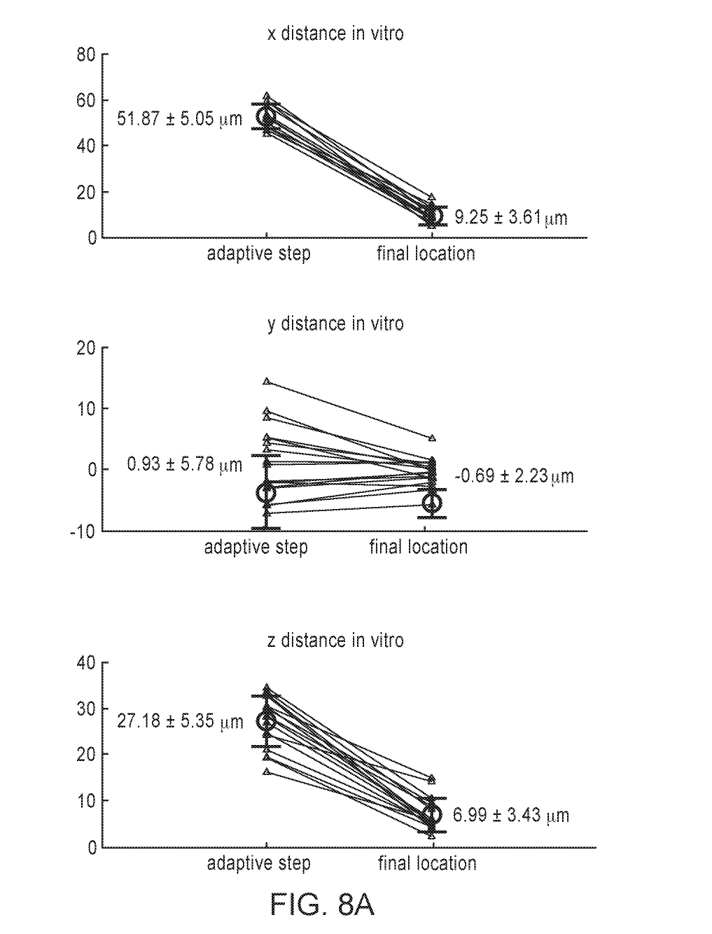

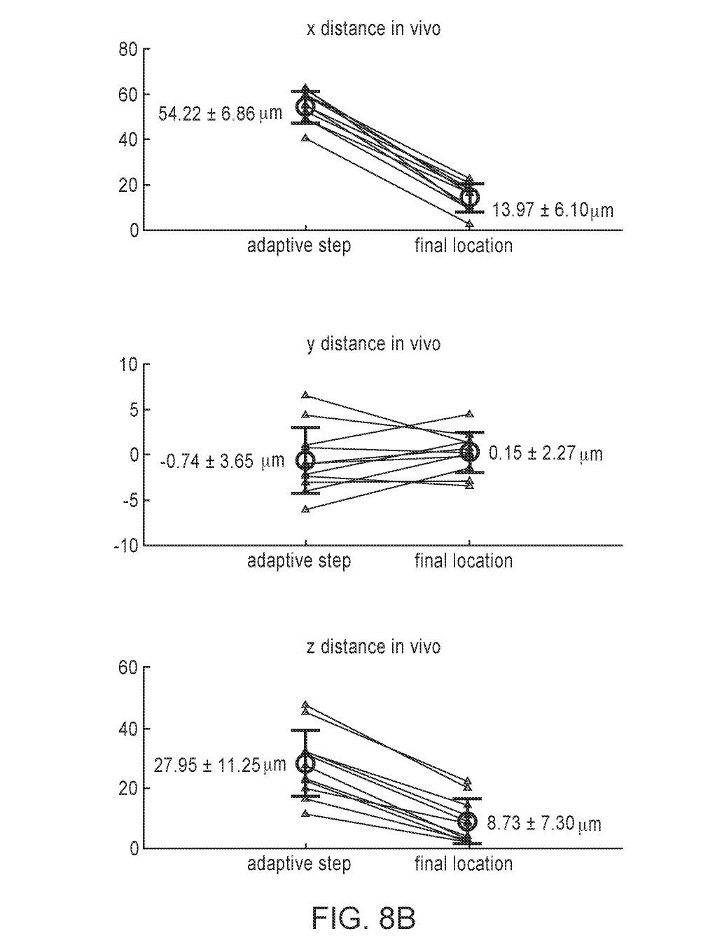

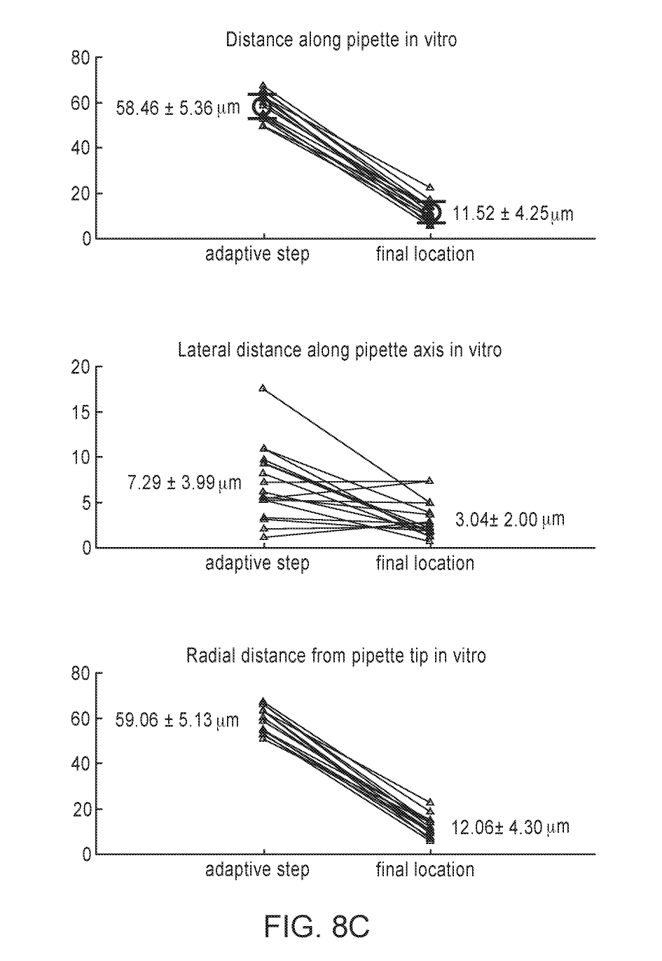

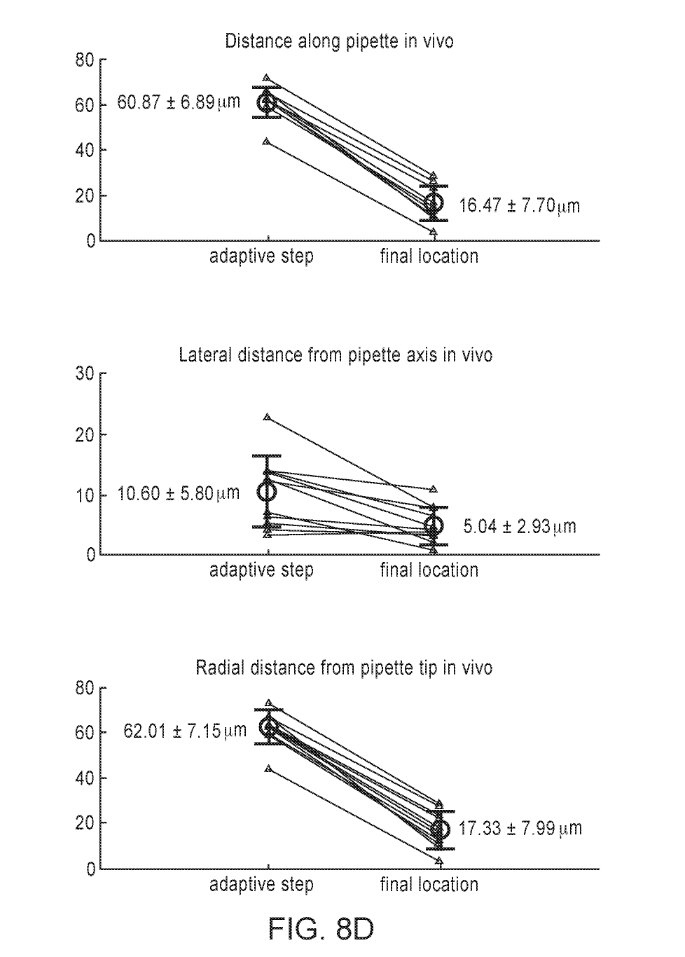

In the adaptive step, a new 3D image substack is collected to scan the z range of the pipette tip and target, automatically locate the tip and target within the substack, and adapt the initial trajectory to compensate for the displacement of the pipette and target. To assess the performance of the method, targeting and approaching tests in vitro and in vivo are performed. The in vitro tests include targeting 2 μm fluorescent beads suspended in agarose, but choosing a target location several microns from the actual target bead and using the adaptive positioning to determine the correct approach to the target. smartACT can reduce the final axial distance from 7.29±2.99 μm at the adaptive step to 3.04±2.00 μm at the final position ˜12 μm from the target, as shown in FIG. 7 and FIGS. 8A-8D.

FIG. 7 shows point cloud of distribution of final pipette positions relative to the target for smartACT approaches in in vitro experiments (blue triangles) and in in vivo experiments (green triangles). The final target position is centered at the origin and the gray sphere has radius of 5 microns to approximate the soma of a pyramidal neuron. The cross-hairs are centered on the mean coordinates and arms indicate standard deviation along and perpendicular to the pipette axis for in vitro and in vivo smartACT approaches, as well as for N=22 non-adaptive approaches.

FIGS. 8A-8D show that adaptive corrections in vivo and in vitro can reduce variability in pipette location. Cartesian coordinates (FIGS. 8A and 8B) and pipette-resolved components (FIGS. 8C and 8D) of the vector between the target position and the pipette tip in vitro (FIGS. 8A and 8C) and in vivo (FIGS. 8B and 8D). Each plot shows the distances at the adaptive step and at the final location, illustrating that the adaptive correction improves lateral distance by ˜2 times.

When applied in vivo to approach targeted neurons in mouse cortex, smartACT's image-based adaptive positioning can reduce deviations. At the end of the approach, smartACT can achieve good alignment of the pipette axis to the target cell body (rₗₐₜₑᵣₐₗ=5.04±2.93 μm, N=11), which is less than half of the average lateral distance measured at the adaptive correction step. Assuming a cell body radius of 5 μm, the final distance from the pipette to the cell surface is 12.33±7.99 μm, indicating that adaptive pipette movements can target the pipette to single neurons in vivo, as shown in FIG. 8A-8D.

To explore the potential of this adaptive step, high signal to noise ratios and image qualities can be helpful to identify both the pipette tip and target cell. In some mouse reporter lines such as Cux2, which can be characterized by relatively dense labeling of pyramidal cells in layer 2/3 with extensive apical arborizations, the background signal from fluorescently labeled dendrites can make segmentation of the pipette tip in the same imaging channel difficult. In order to address this challenge in discriminating the pipette, a green dye (Alexa 488) can be used in the pipette. The green channel can be used for identifying pipette location, while a red channel can be used for visualizing tdTomato fluorescence. In order to further increase fluorescence signal to noise ratio and improve imaging speed, a relatively low pixel resolution 2P image stacks (256×256×N, where N is ca.150 to include pipette tip and target cell) can be used.

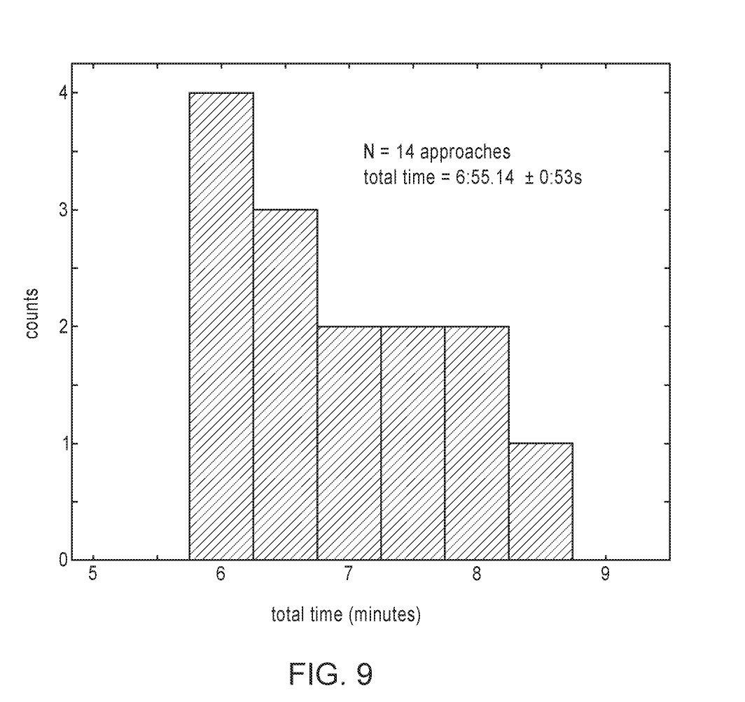

The high degree of accuracy of the final pipette approach provides a repeatable starting position for manual fine adjustments to begin electrophysiology or electroporation experiments. Additionally, the method can take comparable or less time than a manual approach, with the entire adaptive approach process taking 6:55±0:53 min:sec, including approximately 2:30 for image data acquisition, as shown in FIG. 9.

Software Control in the Exemplary Implementation

Initial Targeting:

Initial localization of pipette tip, target cell and pial surface can be done in Vaa3D using single-click virtual finger technology. The planned trajectory includes a retraction step, translation to the entry location (located along a vector parallel to the pipet axis and intersecting the target cell), and termination at a point R distance from the target location along the pipet access. The distance R (typically set at 10-12 microns) is the target buffer distance, supplied in by the user in the interface.

Automatic Pipette Tip and Target Cell Localization:

First, a new z stack (substack) can be collected including the expected pipette tip and target cell locations. From this substack, 3D regions of interest (ROI) around the expected pipette tip and cell locations can be extracted and the actual tip and cell locations can be measured. Specifically, the ROI image data can be background-subtracted and normalized to include the 5^{th}-p^{th} percentile of intensity values independently in the appropriate image channel for tip or cell, where the upper value p may range from 90-99.5, as adjusted in the user interface. The ROI containing the tip can be smoothed with a (3×3×1 pixel) boxcar averaging filter, and the ROI containing the cells can be smoothed using a 2D Gaussian band-pass filter to smooth features larger and smaller than 20 and 2 pixels, respectively. These modified pipette tip and target cell ROIs can be thresholded to segment the pipette tip (green channel) or cell bodies (red channel) into binary image objects based on independent thresholds adjusted in the user interface. The pipette tip can be localized by identifying the mean coordinates of the 10 most anterior voxels of the pipette object in x- y- and z-maximum intensity projections, while the cell body coordinates can be measured as the centroid of segmented cell objects. In case of multiple cells within an ROI, the targeted cell can be identified as the cell whose centroid is closest to the original targeted cell location.

Assessing Precision of Single-Click Targeting:

Two in vivo image volumes and one in vitro image volume (a dilute suspension of 2 μm fluorescent beads in 1.2% low-melt agarose) can be used to assess the precision of single-click targeting using Vaa3D. Each pipette tip or target can be clicked on from a wide range of angles, creating an independent localization attempt for each click. Radial and parallel components of the pipette tip locations in FIGS. 5A-5B can be measured from the line parallel to the nominal pipette axis, through the mean of all pipette localizations.

Experimental Results Using SmartACT

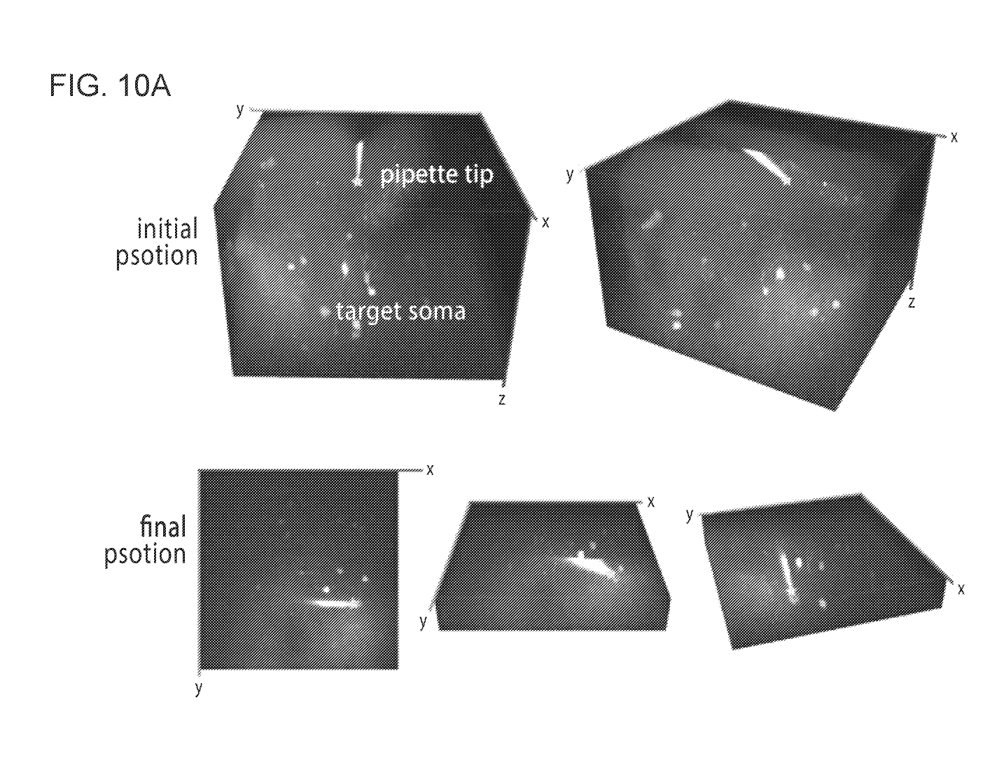

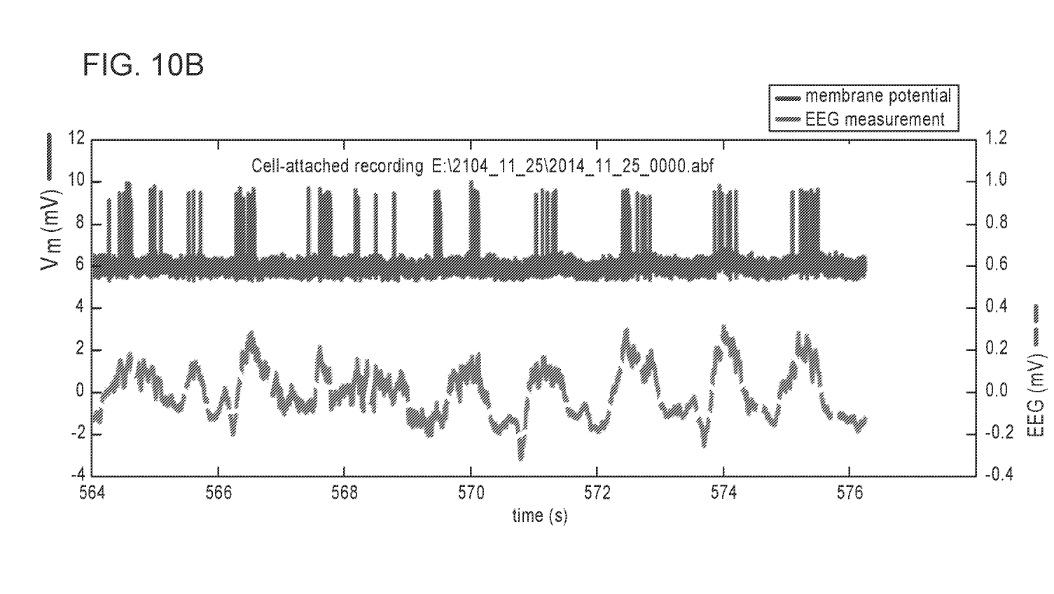

FIGS. 10A and 10B show experimental data acquired using an exemplary smartACT system targeting SST-cre cells in a mouse primary visual cortex. FIG. 10A shows several 3D views of a pipette tip (star) and target location in both initial (top row) and final positions (bottom row). In the final position, the pipette tip is about 11.2 microns from the center of the target cell (11.0 microns axial, 2.2 micros radial). FIG. 10B is a plot of the membrane potential (upper trace) and EEG measurements (bottom trace) made by performing cell-attached clamp current recordings during visual stimulation of the mouse with simultaneous EEG exhibiting synchronization between EEG and SST+ cell firing activity.

Applications of SmartACT to Targeted Single-Cell Experiments

The role for smartACT in biological experiments can be divided into at least four categories, each of which offers potential for interesting scientific advances. First, the smartACT development can be used to facilitate electrophysiological recordings of fluorescently-labeled neurons in the cortex. This category includes cell-attached or juxtacellular recordings, as well as whole-cell recordings across a wide range of experimental paradigms that are often low-efficiency and difficult to standardize.

Second, the same cells targeted for recordings can instead be targeted for intracellular delivery of whatever substances are in the pipette. Current applications of this include combining electrophysiological measurements with plasmid delivery for single-cell protein expression for morphological studies and targeted trans-synaptic labeling. Further applications along these lines include loading of cells with calcium- or voltage-sensitive dyes, other biosensors or even drugs or pathogens for subsequent measurements and perturbations beyond the capabilities of electroporation. The development of smartACT expands the possibilities for other automated experiments targeting single cells in tissue.

Third, smartACT could be deployed to extract cellular contents for cytosolic or nuclear characterization to quantify gene or protein expression levels. When combined with the range of measurements made possible with electrophysiological recordings, single-cell profiling can be used to link genetic and proteomic fingerprint of a cell to its functional role in situ.

The fourth category of potential smartACT applications expands the range of possible targeted experiments by using labeled probes other than a patch pipette. Specifically, if multi-electrode probes, optrodes and GRIN-lens-based micro-endoscopes can be labeled and visualized in 3D, smartACT can be used to adaptively target cells and structures in live tissue for a wide range of measurements and manipulations. These methods could be applied to selectively activate a single cell using optogenetics, measure electrical responses from the vicinity of a specific cell or to target regions for laser microsurgery at the cellular level.

### CONCLUSION

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The above-described embodiments can be implemented in any of numerous ways. For example, embodiments of designing and making the technology disclosed herein may be implemented using hardware, software or a combination thereof. When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

The various methods or processes (e.g., of designing and making the technology disclosed above) outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method of positioning a distal end of a probe with respect to a target location in a tissue, the method comprising:
(A) estimating three-dimensional coordinates of the target location and the distal end of the probe in the tissue from a first image of the tissue;
(B) estimating a path for the distal end of the probe to a desired location in the tissue, the desired location being based at least in part on the three-dimensional coordinates of the target location;
(C) moving the distal end of the probe to within about 25 μm of the three-dimensional coordinates of the target location along the path estimated in (B);
(D) acquiring a second image of the tissue;
(E) estimating, from the second image of the tissue, at least one change in the three-dimensional coordinates of the target location due to insertion and/or movement of the distal end of the probe into the tissue; and
(F) determining at least one change in the path from the distal end of the probe to the desired location in the tissue based at least in part on the at least one change in the three-dimensional coordinates of the target location.

2. The method of claim 1, wherein (B) comprises estimating the path for the distal end of the probe to the desired location in the tissue without accounting for motion of the target location caused by insertion and/or movement of the distal end of the probe into the tissue.

3. The method of claim 1, wherein:
the target location comprises a center of a cell in the tissue, and
(B) comprises selecting the desired location to be located within a predetermined distance from the center of the cell.

4. The method of claim 1, wherein (C) comprises:
inserting the distal end of the probe into the tissue;

5. The method of claim 1, wherein (D) comprises sensing fluorescence generated by stimulating at least one fluorophore in the tissue.

6. The method of claim 1, wherein (D) comprises imaging the target location and the distal tip of the probe in the second image.

7. The method of claim 1, wherein (D) comprises acquiring the second image at a spatial resolution of equal to or finer than about 2 μm.

8. The method of claim 1, further comprising:
(G) moving the distal end of the probe to the desired location at least partially along the path changed in (F).

9. The method of claim 8, wherein (G) comprises aligning the distal end of the probe to within about 5 μm in a lateral dimension and about 8 μm in an axial dimension of the desired location.

10. The method of claim 8, wherein performing steps (A) through (G) occurs within about 10 minutes.

11. The method of claim 8, further comprising at least one of:
measuring an electrical signal at the distal end of the probe;
emitting matter into the tissue via the distal end of the probe; or
withdrawing matter from the tissue via the distal end of the probe.

12. A system comprising:
a probe having a distal end to be inserted into a tissue;
an actuator, mechanically coupled to the probe, to move the distal end of the probe along a predetermined path to a desired location in the tissue, the desired location being within about 25 μm of a target location within the tissue;
an imager to acquire an image of the target location; and
a processor, operably coupled to the actuator and to the imager, to estimate a change in position of the target location caused by insertion and/or movement of the distal end of the probe into the tissue and to determine at least one change in the predetermined path from the distal end of the probe to the desired location in the tissue based at least in part on the change in position of the target location.

13. The system of claim 12, wherein the processor is configured to estimate the predetermined path for the distal end of the probe to the desired location in the tissue without accounting for motion of the target location caused by insertion and/or movement of the distal end of the probe into the tissue.

14. The system of claim 12, wherein:
the target location comprises a center of a cell in the tissue, and
the processor is configured to select the desired location located a predetermined distance from the center of the cell.

15. The system of claim 12, wherein the imager is configured to sense fluorescence generated by stimulating at least one fluorophore in the tissue.

16. The system of claim 12, wherein the imager is configured to image the target location and the distal tip of the probe.

17. The system of claim 12, wherein the imager is configured to acquire the image at a spatial resolution of equal to or finer than about 2 μm.

18. The system of claim 12, wherein the actuator is configured to move the distal end of the probe to the desired location along the path changed by the processor.

19. The system of claim 18, wherein the actuator is configured to align the distal end of the probe to within about 5 μm in a lateral dimension and about 8 μm in an axial dimension of the desired location.

20. The system of claim 18, wherein the system is configured to move the distal end of the probe to the desired location within about 10 minutes of inserting the probe into the tissue.

21. The system of claim 18, wherein the probe is configured to:
sense an electrical signal at the distal end of the probe;
emit matter into the tissue via the distal end of the probe; and/or
withdraw matter from the tissue via the distal end of the probe.
